(19) United States
(12) Patent Application Publication
(10) Pub. No.: US 2002/0107171 A1
(43) Pub. Date: Aug. 8, 2002

(54) Regulation of the P21 gene and uses thereof

(76) Inventors: Peter M. Price, Little Rock, AR (US); Judit Megyesi, Little Rock, AR (US); Robert Safirstein, Little Rock, AR (US)

Correspondence Address: Benjamin Aaron Adler, ADLER & ASSOCIATES, 8011 Candle Lane, Houston, TX, 77071, US

(21) Appl. No.: 09/881,635

(22) Filed: Jun. 14, 2001

Related U.S. Application Data
(60) Provisional application No. 60/212,224, filed on Jun. 16, 2000.

(51) Int. Cl. A61K 31/00

(52) U.S. Cl. 514/1

(57) ABSTRACT

The present invention provides a method for treating or preventing a pathophysiological state of an organ in an individual wherein this state is characterized by an undesirable level of cyclin-dependent kinase inhibitor activity in the organ, comprising the step of regulating the expression of the p21 gene in the organ of the individual. Also provided is a method for treating chronic progressive renal failure in an individual in need of such treatment, comprising the step of regulating the expression of the p21 gene in one or both kidneys of the individual wherein the regulation of the p21 gene results in the manipulation of cyclin-dependent kinase inhibitor activity in one or both kidneys. In addition there is provided a method of lowering the rate of long-term rejection of a transplanted organ in an individual comprising the step of transplanting into the individual the organ from a donor wherein the p21 gene in the organ is not expressed.

### CROSS-REFERENCE TO RELATED APPLICATION

This non-provisional patent application claims benefit of provisional patent application U.S. Serial No. 60/212,224 filed Jun. 15, 2000, now abandoned.

### FEDERAL FUNDING LEGEND

This invention was produced in part using funds obtained through grant R01 DK54471 from the National Institutes of Health. Consequently, the federal government has certain rights in this invention.

### BACKGROUND OF THE INVENTION

1. Field of the Invention

The present invention relates generally to the fields of molecular biology and organ transplantation. More specifically, the present invention relates regulation of the expression of the p21 gene to treat chronic failure and/or rejection of organs.

2. Description of the Related Art

The removal of substantial amounts of renal tissue is followed by a progressive decline in renal function (1, 2). Glomerular hypertrophy occurs early in response to this ablation and is accompanied by short-term increases in glomerular filtration (3, 4). These structural and functional adaptations to loss of excretory function are thought to be maladaptive and to influence the progression to end stage renal disease. Progression is initially seen as localized increases in mesangial matrix that then leads to global glomerular sclerosis, and is usually associated with systemic hypertension, which has been speculated to accelerate its course. Although the early glomerular hypertrophy and hyperfunction, especially the glomerular hypertension that determines it, have been invoked as predeterminants of the later destructive effects of renal ablation, there is no established causal link between these events and the progressive nature of the renal disease.

Acute short-term stress in the kidney provokes molecular responses that involve the expression of several genes, including the cyclin-dependent kinase (cdk) inhibitor p21(5). p21 plays a critical role in processes by which nuclear events subsequent to environmental stress are regulated. p21 is induced to very high levels by oxidative stress (6) and DNA damage (7). The p21 protein (8) acts as an inhibitor of cyclin-dependent kinase activity (9) and effectively stops cell-cycle progression (8, 9). p21 is over expressed in many cells undergoing senescence (10) or terminal differentiation (11, 12). The expression of p21 following short term chemotoxic renal stress is rapid and expression of p21 under these circumstances played a protective role (13). Chronic, long term stress could provoke sustained expression of p21 and that such expression could influence renal function and morphology. Controlling p21 function may ameliorate or even prevent progressive end-stage renal disease or other pathophysiological states in other organs.

The prior art is deficient in the lack of gene regulation to treat chronic organ failure. The present invention fulfills this long-standing need and desire in the art.

### SUMMARY OF THE INVENTION

One object of the present invention is a method for treating or preventing a pathophysiological state of an organ in a n individual wherein this state is characterized by an undesirable level of cyclin-dependent kinase inhibitor activity in the organ, comprising the step of regulating the expression of p21 in the organ of the individual.

Another object of the present invention is a method for treating chronic progressive renal failure in an individual in need of such treatment, comprising the step of regulating the expression of p21 in one or both kidneys of the individual wherein the regulation of p21 results in the manipulation of cyclin-dependent kinase inhibitor activity in one or both kidneys.

Yet another object of the present invention is a method of lowering the rate of long-term rejection of a transplanted organ in an individual comprising the step of transplanting into the individual the organ from a donor wherein the p21 gene in the organ can not be expressed.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows renal function following ablation. Clearance of inulin (ml per minute) is calculated per gram kidney is calculated in mice from both genotypes. Statistically significant differences are only noted between the two populations at 14-16 weeks after ablation (p=0.04). Values shown in the figure represent ± standard error.

FIG. 2 shows the mean arterial pressure. Mean systolic blood pressure is obtained by catheterizing the left femoral artery. Statistically significant differences between the two populations is noted as early as 6-8 weeks after ablation (p=0.005), which increases by 14-16 weeks after ablation (p=0.00002). Values represent ± standard error.

FIG. 3 shows the histologic changes in remnant kidney after ablation. Representative sections from either untreated (FIGS. 3A and 3B), 8 week (FIGS. 3C and 3D), 16 week (FIG. 3E), or 26 week (FIG. 3F) after ablation of wild-type (FIGS. 3A, 3C, and 3E) or p21(−/−) mice (FIGS. 3B, 3D, and 3F). X390. Sections are stained with periodic acid-Schiff (PAS).

FIG. 4 shows the detection of interstitial fibrosis using trichrome stain in remnant kidney after ablation. Representative sections from either 6 week (FIG. 4A) or 26 week (FIG. 4B) after ablation of wild-type (FIG. 4A) or p21(−/−) mice (FIG. 4B) X390.

FIG. 5 shows the in situ hybridization for localization of p21 mRNA in remnant kidney cells after partial renal ablation. Hybridization of an antisense p21 probe to RNA in cells of remnant kidney 4 weeks (FIG. 5A) and 14 weeks (FIG. 5B) after ablation. X390.

FIG. 6 shows the cell cycle analysis in remnant kidney cells after partial renal ablation. Immunodetection of nuclear PCNA localization 2 weeks after ablation in kidney sections from p21(−/−) (FIG. 6A) and wild-type mice (FIG. 6B). X390.

### DETAILED DESCRIPTION OF THE INVENTION

In one embodiment of the present invention there is provided a method for treating or preventing a pathophysiological state of an organ in an individual wherein said state is characterized by an undesirable level of cyclin-dependent kinase inhibitor activity in said organ, comprising the step of regulating the expression of the p21 gene in said organ of said individual. Preferably, the organs of treatment are the kidneys, heart, liver, lungs, and other organs amenable to transplantation. Representative examples of pathophysiological states are renal fibrosis, glomerulosclerosis, reduced filtration rates, hypertension and organ transplantation rejection. In one aspect of this embodiment the regulation of the expression of p21 results in the reduction or elimination of p21 expression. Preferably, reduction or elimination or p21 expression is performed by a techniques such as drug therapy, genetic manipulation, antisense DNA, etc. In another embodiment, the present invention is directed to a method for treating chronic progressive renal failure in an individual in need of such treatment, comprising the step of regulating the expression of p21 in one or both kidneys of the individual wherein the regulation of p21 results from the manipulation of cyclin-dependent kinase inhibitor activity in one or both kidneys.

In yet another embodiment of the present invention, there is provided a method of lowering the rate of long-term rejection of a transplanted organ in an individual in need of such treatment comprising the step of transplanting into the individual the organ from a donor wherein the p21 gene in the transplanted organ is not expressed.

The following definitions are given for the purpose of facilitating understanding of the inventions disclosed herein. Any terms not specifically defined should be interpreted according to the common meaning of the term in the art.

As used herein, the term “individual” shall refer to animals and humans.

Partial renal ablation leads to progressive renal insufficiency and is a model of chronic renal failure from diverse causes. Mice develop functional and morphologic characteristics of chronic renal failure after partial renal ablation including glomerular sclerosis, systemic hypertension and reduced glomerular filtration. However, litter-mates having a homozygous deletion of the gene for the cyclin-dependent kinase inhibitor, p₂^{WAF1/CIP1}, do not develop chronic renal failure after ablation. The markedly different reactions of the p21(+/+) and p21(_/_) animals was not due to differences in glomerular number or degree of renal growth, but rather to the presence or absence of a normal p21 gene. While the reaction to the stress of renal ablation is both hyperplastic and hypertrophic in the presence of a functional p21 gene, the absence of the p21 gene may induce a more hyperplastic reaction since PCNA expression, a marker of cell-cycle progression, in the renal epithelium of the remnant kidney is more than five times greater in the p21(−/−) mice than in the p21(+/+) animals. As p21 is a potent inhibitor of the cell-cycle, p21 may regulate the balance between hyperplasia and hypertrophy following renal ablation. This change in response inhibits the development of chronic renal failure.

The following examples are given for the purpose of illustrating various embodiments of the invention and are not meant to limit the present invention in any fashion.

### EXAMPLE 1

Animal Preparation

Mice (strain 129/Sv) carrying a deletion of a large portion of the p21 gene in which neither p21 mRNA nor p21 protein is expressed (14) were obtained from Dr. Philip Leder (Harvard Medical School, Cambridge, Mass.). Mice homozygous for the p21 deletion are selected from the offspring of heterozygous matings using Southern blotting of tail DNA as described (14). Wild-type p21(+/+) litter-mates are used as controls for a normal p21 gene. The animals are housed at the Animal Research Center at the University of Texas Medical Branch at Galveston. Food and water are supplied ad libitum. Body weights are determined at the start of the protocol, at the time of surgery, and at the time of sacrifice.

Renal ablation is created by two-step nephrectomy (15) using 6-8 week-old male mice. At the first stage of the procedure, the right kidney is decapsulated and the upper and lower poles are resected under anesthesia with Pentobarbital Sodium (50 mg/Kg) ip. Bleeding is prevented using a thrombin solution (3000 units/ml 0.9% NaCl). One week later, a total left nephrectomy is performed under anesthesia as described above. Renal function, kidney morphology, morphometry and mean arterial blood pressure are studied a t various times thereafter.

### EXAMPLE 2

Clearance and Direct Systolic Blood Pressure Measurements

Mice are anesthetized, as above, and placed on a heated surgical table to maintain body temperature between 37-38° C. Polyethylene catheters are placed in the trachea, bladder, both femoral arteries and left jugular vein. The mean arterial blood pressure is obtained via the left femoral artery using a strain-gauge transducer (Gould, Cleveland, Ohio). The animals are infused with 0.9% sodium chloride solution via the left external jugular vein at a rate of 0.5% body weight/hour using a constant infusion syringe pump (Model 355, Sage Instruments, Cambridge, Mass.). The infusion solution containes enough [Methoxy-³H]-inulin (American Radiolabeled Chemicals, St. Louis, Mo.) to deliver 10 μCi/hour. After a 60 minute equilibration period, urine is collected under mineral oil for three 30 minute clearance determinations. Blood is drawn in heparinized microhematocrit tubes from the right femoral artery a t the beginning and end of the clearance period to determine hematocrit and [³H] activity. [³H] activity in urine and plasma is determined in a liquid scintillation counter (LKB Wallace 1211 RackBeta) and the GFR calculated.

### EXAMPLE 3

Kidney Morphology and Morphometry

At the time of sacrifice, kidney remnants are freed from the surrounding tissues, weighed and cut in half, fixed in 4% neutral buffered formaldehyde, and processed for light microscopy by paraffin embedding. Sections (5 μm) are stained with hematoxylin-eosin, periodic-acid Schiff (PAS) or trichrome.

Morphological Studies

Three to five animals at various time points are used for morphological studies. Using PAS-stained sections, at least 300 glomeruli are evaluated by light microscopy. The percentage of each glomerulus exhibiting mesangial expansion or glomerulosclerosis was determined by point counting (4) at x400 using an eyepiece reticle (S075963, Nikon Inc.) Focal glomerulosclerosis is graded as to percent of glomerular area sclerotic using the following criteria: minimal (1-25%), moderate (26-50%) and severe (51-84%). When ˜85% of glomerular area is sclerotic, the glomerulus is classified as globally sclerotic.

Glomerular Morphometry

To determine glomerular hypertrophy mean glomerular volume (MGV, μ m³) is measured based on point counting (16-18) according to the following formula:

$$MGV = {1.25 [ ( antilog \frac{log P}{n} ) {k}^{2} ]}^{3 / 2}$$

P=number of points falling on each glomerular tuft profile

k=distance between the points in micrometers

n=number of glomeruli counted

Glomeruli showing global sclerosis were excluded.

### EXAMPLE 4

Quantitation of Glomerular Numbers Per Kidney

The number of glomeruli per kidney was determined by using the method described by MacKay et al (19).

### EXAMPLE 5

Immunohistochemistry

Proliferating cell nuclear antigen (PCNA) is detected using a mouse monoclonal antibody (Santa Cruz Laboratory, Santa Cruz, Calif.) and the ABC Elite Vectastain Kit (Vector Laboratories, Inc., Burlingame, Calif.), according to manufacturers instructions.

### EXAMPLE 6

In situ Hybridization

In situ localization of p21 mRNA on kidney sections was performed as previously described (5).

### EXAMPLE 7

Calculations: GFR

C_{Inulin}(ml/min)=U/P [³H]×Vu (ml/min)

Percent (%) Nephrectomy and Hypertrophy

$$\% nephrectomy = \frac{{RK}_{REMOVED} + {LK}_{ADJ}}{2 \times {LK}_{ADJ}} \times 100$$

where RK_{REMOVED} is the amount (in mg) of the right kidney removed in the first operation; and LK_{ADJ} is the weight (mg) of the left kidney removed in the second operation 7 days later, adjusted for hypertrophy between the first and second operation. The adjustment is calculated by multiplying the weight of the left kidney at the time of removal by the average kidney weight per body weight of untreated animals divided by the average kidney weight per body weight of day 7 left kidneys.

$$\% hypertrophy = \frac{{RK}_{FINAL} - {RK}_{INTACT}}{{RK}_{INTACT}} \times 100$$

where RK_{FINAL} was the weight (mg) of right kidney at sacrifice; and RK_{INTACT} is LK_{ADJ} RK_{REMOVED}.

### EXAMPLE 8

Statistical Analysis

Results are presented as means ±SE. Differences between means are evaluated using the Student's t-test for unpaired data. p<0.05 is considered statistically significant.

### EXAMPLE 9

Body Weight and Renal Parameters before Ablation

Body weight, kidney weight, glomerular number and volume, and renal function in untreated p21(+/+) and (−/−) mice are given in Table 1. There are no phenotypic differences between the two groups of mice, although the untreated p21(−/−) animals are about 15% (p<0.001) larger than those in the p21(+/+) group. Size increases have also been reported in mice lacking the p27 cdk inhibitor genes (20-23). However, neither kidney weight per gram body weight, total glomerular number, nor mean glomerular volume are different between the two genotypes. Similarly, the two-kidney glomerular filtration rate (GFR, expressed as C^{inulin}) of the untreated animals is not different.

TABLE 1
Physical Parameters in Untreated Mice

| | | Kidney | Number | Mean | |
|---|---|---|---|---|---|
| | | Weight | of | Glomerular | |
| | Body | (mg/g | Glomeruli | Volume x | Cᵢₙᵤₗᵢₙ |
| | Weight | body | per | 10-5 | (ml/ |
| | (g) | weight) | Kidney | (μn3) | min) |
| p 21 | 24.35 ± | 5.938 ± 0.66 | 12583 ± | 1.92 ± 0.58 | 1.09 ± |
| (+/+) | 2.68 | | 681 | | 0.07 |
| p 21 | 28.47 ± | 5.720 ± 0.61 | 12091 ± | 1.74 ± 0.1 | 1.05 ± |
| (_/_) | 4.18 | | 555 | | 0.08 |
| p | p <0.001 | NS | NS | NS | NS |
| value | | | | | |

Body weight, kidney weight, glomerular number, glomerular volume, and GFR in untreated mice. Values are means ± standard deviation.
NS = not significant.

### EXAMPLE 10

Body Weight, Degree of Ablation, Remnant Hypertrophy and Mean Glomerular Volume after Ablation

Weight gain in renal ablated mice throughout the 14-16 week period of observation was not significantly different between the two groups, either in absolute terms (2.3±0.8 g vs 4.3±1.1 g; +/+vs −/− groups, respectively; n=11 in each group) or relative to initial body weight (10.2±3.5% vs 15.9±4.3%; +/+vs −/− groups, respectively). The degree of renal ablation was determined for each genotype. Approximately ⅔ of the normal renal mass w as removed after the 2 operations and there is no significant difference between the groups. The percent nephrectomy in the p21(+/+) and p21(−/−) groups was 68.8±3.6% and 68.3±3.1% (p=0.619), respectively. Furthermore, the degree of hypertrophy and the mean glomerular volume after ablation (Table 2) was not significantly different between the groups.

TABLE 2
Hypertrophy (%) MGV × 10₋₅ (μm³)

| Weeks | p21 | p21 | t-test | p21 (+/+) | p21 | t-test |
|---|---|---|---|---|---|---|
| | (+/+) | (_/_) | | | (_/_) | |
| Control | NA | NA | NA | 1.92 ± 0.58 | 1.74 ± | NS |
| | | | | | 0.14 | |
| 2-4 W | 66.9 ± | 83.9 ± 85.7 | NS | 1.93 ± 0.32 | 2.58 ± | NS |
| | 28.6 | | | | 0.71 | |
| 6-8 W | 86.8 ± | 138.5 ± 55.8 | NS | 2.71 ± 0.33 | 2.99 ± | NS |
| | 41.5 | | | | 0.34 | |
| 10-12 W | 137.1 ± | 141.4 ± 31.3 | NS | 3.52 ± 0.21 | 3.34 ± | NS |
| | 88.7 | | | | 0.37 | |
| 14-16 W | 145.0 ± | 135.9 ± 42.1 | NS | 2.87 ± 0.50 | 3.17 ± | NS |
| | 37.0 | | | | 0.38 | |

Precent hypertrophy and mean glomerulr volumes after renal ablation. Values are means ± standard deviation.
NS = not significant; NA = not applicable.

### EXAMPLE 11

Renal Function following Ablation

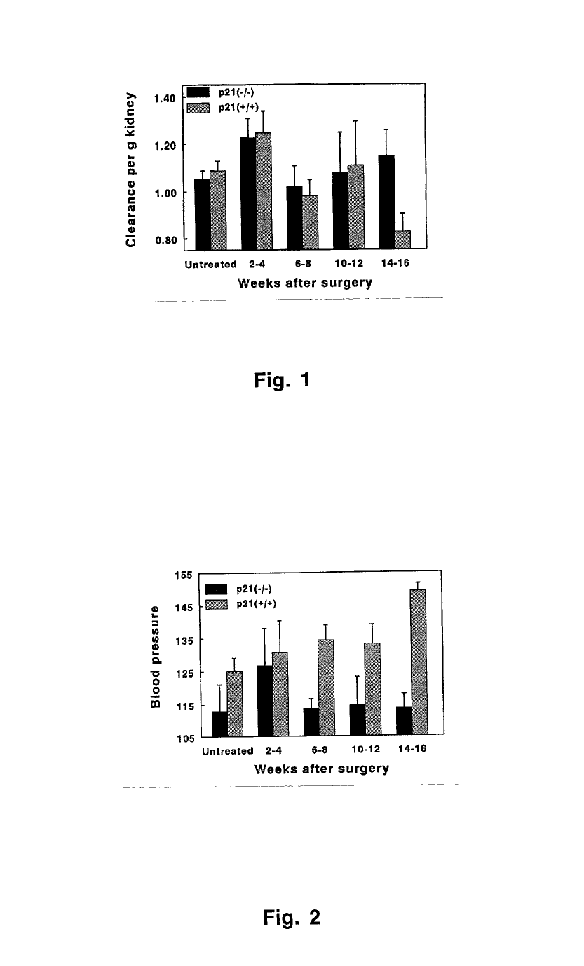

Glomerular filtration rate increased to the same extent 2 to 4 weeks after ablation in both groups. Glomerular filtration rate was similar in both groups until the 14^{th}-16^{th} week after ablation when it falls in the wild-type animals but remains unchanged from previous values in the p21(_/_) group. The glomerular filtration rate at this time point was significantly different between the two groups (p<0.05) (FIG. 1).

### EXAMPLE 12

Mean Arterial Pressure

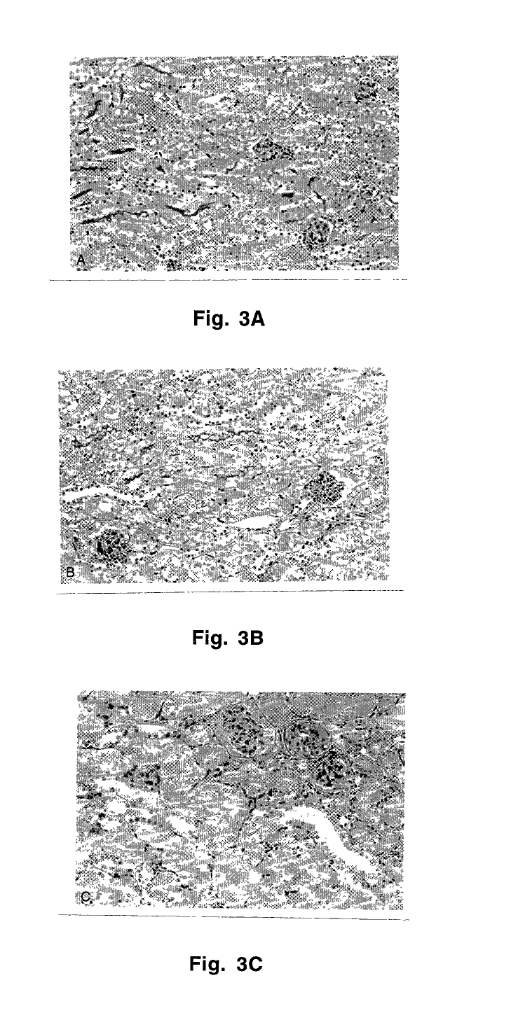

Mean arterial pressure is not significantly different between the untreated groups of animals. Following partial renal ablation, arterial pressure increases initially in both groups of animals and increases further in the p21(+/+) mice so that by the 14^{th}-16^{th} week the average mean systolic pressure reaches 150.7±6.7 mm Hg (mean ±SD). By contrast, mean systolic blood pressure in the p21(−/−) mice returns toward normal and remains there throughout the 16-week period of observation (113.8±17.7 after 16 weeks versus 112.8±16.7 in untreated mice) (FIG. 2).

### EXAMPLE 13

Morphology

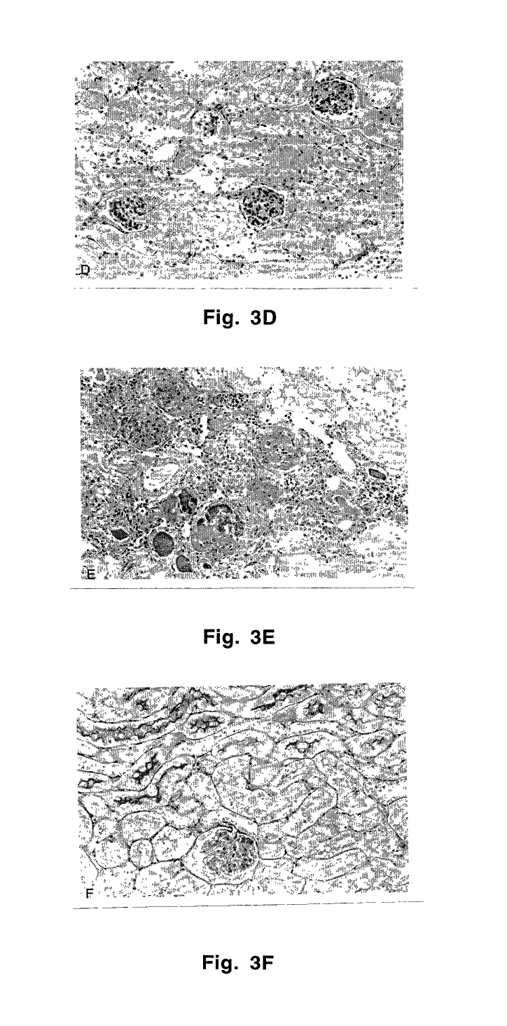

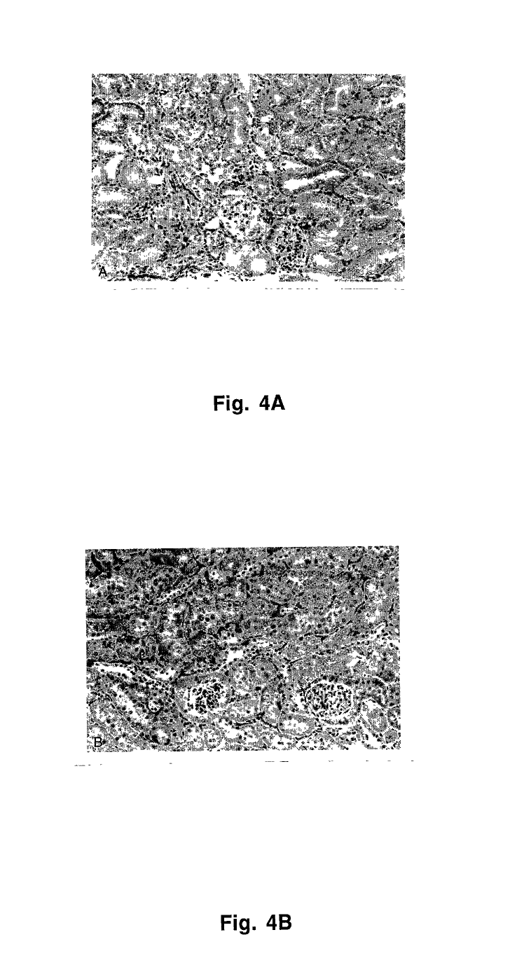

Light microscopic study reveals a marked difference of histologic changes between the two groups of mice. Representative micrographs are given in FIGS. 3 and 4; the changes are quantified in Table 3. Kidney sections from untreated mice were morphologically indistinguishable (FIGS. 3A, 3B). Mesangial expansion and mild focal glomerulosclerosis was observed in about 70% of glomeruli in the p21(+/+) mice 4 weeks after ablation (Table 3). Beginning at 6 to 8 weeks these mice developed severe focal and global glomerulosclerosis (FIG. 3C [cf FIG. 3D], FIG. 4A, Table 3). All of the p21(+/+) mice studied developed glomerulosclerosis accompanied by interstitial fibrosis and round cell infiltration by 14-16 weeks post ablation (FIG. 3E, Table 3). In contrast, p21(−/−) mice never developed glomerulosclerosis nor interstitial changes even 26 weeks after renal ablation (FIG. 3F, FIG. 4B) although mesangial expansion was seen occasionally.

The percentages of glomerulosclerosis in the p21(+/+) mice at various times after ablation are quantified in Table 3. It can be seen that they developed a progressive increase in glomerular sclerosis. The p21(−/−) mice do not develop glomerulosclerosis throughout the period of observation and were omitted from the table.

TABLE 3
Glomeruloscierosis in p21 (+/+) Mice

| Weeks | None | Minimal | Moderate | Severe | Global |
|---|---|---|---|---|---|
| 4 W | 30.7 ± 0.9 | 65.8 ± 1.6 | 2.8 ± 1.7 | 0.8 ± 0.7 | 0 |
| 6-8 W | 27.8 ± 5.1 | 41.0 ± 2.7 | 22.2 ± 3.5 | 4.9 ± 1.8. | 4.2 ± 3.5 |
| 10-12 W | 15.3 ± 3.2 | 38.2 ± 7.8 | 25.2 ± 3.5 | 10.2 ± 3.3 | 11.1 ± 6.8 |
| 14-16 W | 23.6 ± 2.5 | 22.4 ± 4.4 | 36.5 ± 4.1 | 9.5 ± 1.9 | 8.1 ± 3.9 |

Development of glomerulosclerosis in p21 (+/+) mice. Percent glomeruli in each category (± standard error) as defined in Methods section.

### EXAMPLE 14

Expression of p21 in the Remnant Kidney

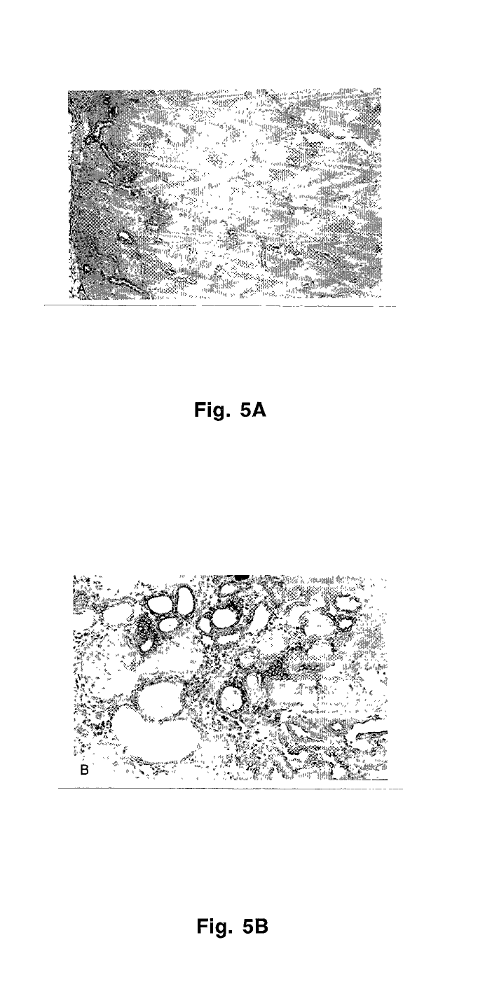

In situ hybridization for p21 mRNA identifies the cells of the cortical thick ascending limbs and distal convoluted tubules as the principal site of p21 expression 4 weeks following ablation (FIG. 5A). At later times, it was also expressed in the epithelium of tubules (primarily dilated and collapsed) and glomeruli within or adjacent to sclerotic areas of the remnant kidney (FIG. 5B).

### EXAMPLE 15

Cell Cycle Analysis

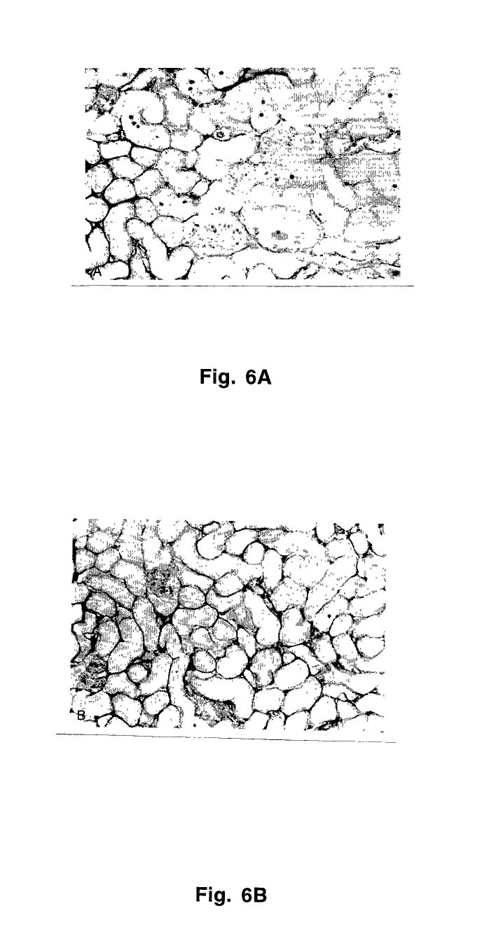

Nuclear PCNA, a marker for cells in the S phase of the cell cycle is found in many cells of the remnant kidney in the p21(−/−) mice 2 weeks after surgery (FIG. 6A). The positive nuclei are primarily localized in the proximal convoluted tubules and occasionally in the glomeruli and distal convoluted tubules. By contrast, few cell nuclei are stained in the p21(+/+) remnant kidney (FIG. 6B). This difference in PCNA staining is quantified in nuclei from p21(−/−) mice (18.64±0.73 per mm²) and p21(+/+) mice (3.50±0.65 per mm²) and is highly significant (p=0.00006). At later time points, PCNA is greatly diminished in both animals (data not shown).

Discussion

Mice lacking a p21 gene were resistant to the functional and morphologic consequences of partial renal ablation. Not only is the resistance manifested locally in the surgically impaired remnant organ, but it is also evident systemically in the lack of increased arterial pressure. This resistance may be due to several parameters that may be early determinants of the long-term outcome of renal ablation. Severe protein restriction can partially ameliorate the development of glomerulosclerosis after partial renal ablation (24). However, weight gains in the two groups of animals is not significantly different, and the p21(_/_) mice even experience slightly elevated gains, both relative and absolute. Reduced glomerular number may be an etiologic link in the progressive nature of renal disease (25, 26). The p21(+/+) and (−/−) animals have similar numbers of glomeruli at the outset of the experiments (Table 1) and the degree of renal ablation is the same for each group. Thus the loss of renal excretory function is equally applied to both groups. The increase in glomerular filtration that occurs in response to renal ablation, also thought to be an early determinant of the progression (4), occurs to the same extent in the p21(_/_) animals as it does in the wild type (FIG. 1). Glomerular hypertrophy, which has a n independent role in the progression of renal ablation models of experimental renal disease (27), occurs to the same extent in both groups as well (Table 2).

Taken together, this indicates that the p21 gene product plays a critical role in the functional and morphologic consequences subsequent to the stress of renal ablation, including the development of glomerular sclerosis and hypertension. Additionally, hypertension does not develop without the development of renal damage. This resistance may be critically linked to the prominent role the p21 protein (cdk) plays in regulating the cell cycle. The growth of the kidney following renal ablation is a consequence of hyperplasia and hypertrophy of the glomerular and epithelial compartments of the kidney (28, 29). However, hypertrophy may be in the long term, a maladaptive response to the loss of functional renal tissue (4, 27, 30).

In the absence of the p21 gene the growth response of the kidney after partial ablation is relatively more hyperplastic than hypertrophic. Consistent with this notion is a greater than 5-fold increase in PCNA protein expression in p21(_/_) animals compared to the wild-type animals undergoing the response to renal ablation. By achieving growth after renal ablation by increasing the relative contribution of hyperplasia, the work load of the kidney is better accommodated. This assumes that when an organ accommodates increases in work by hypertrophy rather than hyperplasia, it is at a serious physiologic disadvantage and more likely to undergo regression of structure and function (31). A detailed description of the differences in the balance between hypertrophy and hyperplasia in the two groups of mice and, more specifically, the sites at which these differences are apparent would confirm this assumption. It is clear that p21 is a critical sensor of the stress of renal mass reduction. This model may be useful in identifying the mechanism of how this response to renal ablation is maladaptive. The studies also suggest that manipulation of p21 gene expression could be a target for the treatment of progressive renal failure.

The following references are cited herein:

Chanutin, A. & Ferris, E. B., Jr. (1932) Experimental renal insufficiency produced by partial nephrectomy. I. Control diet. Arch. Intern. Med. 49, 767-787.

Morrison, A. B. (1962) Experimentally induced chronic renal insufficiency in the rat. Lab Invest. 11, 321-332.

Faraj, A.H. & Morley, A. R. (1992) Remnant kidney pathology after five-sixth nephrectomy in rat. I. A biochemical and morphological study. APMIS 100, 1097-1105.

Daniels, B. S. & Hostetter, T. H. (1990) Adverse effects of growth in the glomerular microcirculation. Am. J. Physiol. 258, F1409-F1416.

Megyesi, J., Udvarhelyi, N., Safirstein, R. L., & Price, P. M. (1996) The p53-independent activation of transcription of p21^{WAF1/CIP1/SDI1} after acute renal failure. Am. J. Physiol. 271, F1211-F1216.

Gorospe, M., Martindale, J. L., Sheikh, M. S., Fornace, A. J., Jr., & Holbrook, N. J. (1996) Regulation of p₂₁^{CIP1/WAF1} expression by cellular stress: p53-dependent and p53-independent mechanisms. Mol. Cell. Differ. 4, 47-65.

El-Deiry, W. S., Tokino, T., Velculescu, V. E., Levy, D. B., Parsons, R., Trent, J. M., Lin, D., Mercer, W. E., Kinzler, K. W., & Vogelstein, B. (1993) WAF-1, a potential mediator of p53 tumor suppression. Cell 75, 817-825.

Xiong, Y., Zhang, H. & Beach, D. (1992) D type cyclins associate with multiple protein kinases and the DNA replication and repair factor PCNA. Cell 71, 505-514.

Harper, J. W., Adami, G. R., Wei, N., Keyomarsi, K. & Elledge, S. J. (1993) The p21 cdk-interacting protein Cip1 is a potent inhibitor of G1 cyclin-dependent kinases. Cell 75, 805-816.

Noda, A., Ning, Y., Venable, S. F., Pereira-Smith, O. M. & Smith, J. R. (1994) Cloning of senescent cell-derived inhibitors of DNA synthesis using an expression screen. Exp. Cell Res. 211, 90-98.

Steinman, R. A., Hoffman, B., Iro, A., Guillouf, C., Liebermann, D. A. & el-Houseini, M. E. (1994) Induction of p21(WAF-1/CIP1) during differentiation. Oncogene 9, 3389-3396.

Jiang H., Lin, I., Su, Z. Z., Collart, F. R., Huberman, E., & Fisher, P. B. (1994) Induction of differentiation in human promyelocytic HL-60 leukemia cells activates p21, WAF1/CIP1, expression in the absence of p53. Oncogene 9, 3397-3406.

Megyesi, J., Safirstein, R. L. & Price, P. M. (1997) Induction of p21^{WAF1/CIP1/SDI1} in kidney tubule cells affects the course of cisplatin-induced acute renal failure. J. Clin. Invest. 101, 777-782.

Deng, C., Zhang, P., Harper, J. W., Elledge, S. J., & Leder, P. (1995) Mice Lacking p21^{CIP1/WAF1} undergo normal development, but are defective in G1 checkpoint control. Cell 82, 675-684.

Hamamori, Y., Samal, B., Tian, J. & Kedes, L. (1995) Myoblast transfer of human erythropoietin gene in a mouse model of renal failure. J. Clin. Invest. 95, 1808-1813.

Nath, K. A. & Salahudeen, A. K. (1990) Induction of renal growth and injury in the intact rat kidney by dietary deficiency of antioxidants. J. Clin. Invest. 86, 1179-1192.

Hirose, K., Østerby, R., Nozawa, M. & Gundersen, H. J. G. (1982) Development of glomerular lesions in experimental long-term diabetes in the rat. Kidney Int. 21, 689-695.

Bilous, R. W., Mauer, S. M., Sutherland, D. E. R. & Steffes, M. W. (1989) Mean glomerular volume and rate of development of diabetic nephropathy. Diabetes 38, 1142-1147.

MacKay, K., Striker, L. J., Pinkert, C. A., Brinster, R. L. & Striker, G. E. (1987) Glomerulosclerosis and renal cysts in mice transgenic for the early region of SV40. Kidney Int. 32, 827-837.

Nakayama, K, Ishida, N., Shirane, M., Inomata, A., Inoue, T., Shishido, N., Horii, I., Loh, D. Y., & Nakayama, K. -i. (1996) Mice lacking p27^{KiP1} display increased body size, multiple organ hyperplasia, retinal dysplasia, and pituitary tumors. Cell 85, 707-720.

Kiyokawa, H., Kineman, R. D., Manova-Todorova, K. O., Soares, V. C., Hoffman, E. S., Ono, M., Khanam, D., Hayday, A. C., Frohman, L. A., & Koff, A. (1996 ) Enhanced growth of mice lacking the cyclin-dependent kinase inhibitor function of p27^{KiP1}. Cell 85, 721-732.

Fero, M. L., Rivkin, M., Tasch, M., Porter, P., Carow, C. E., Firpo, E., Polyak, K., Tsai, L. -H., Broudy, V., Perlmutter, R. M., Kaushansky, K., & Roberts, J. M. (1996) A syndrome of multiorgan hyperplasia with features of gigantism, tumorigenesis, and female sterility in p27^{KiP1}-deficient mice. Cell 85, 733-744.

Franklin, D.S., Godfrey, V. L., Lee, H., Kovalev, G. I., Schoonhoven, R., Chen-Kiang, S., Su, L., & Xiong, Y. (1998) CDK inhibitors p18^{INK4c} and p27^{KiP1} mediate two separate pathways to collaboratively suppress pituitary tumorigenesis. Genes Dev. 12, 2899-2911.

Brenner, B. M., Meyer, T. W., & Hostetter, T. H. (1982) Dietary protein intake and the progressive nature of kidney disease: the role of hemodynamically mediated glomerular injury in the pathogenesis of progressive glomerular sclerosis in aging, renal ablation, and intrinsic renal disease. N. Engl. J. Med. 307, 652-660.

Brenner, B. M., Garcia, D. L. & Anderson, S. (1988) Glomeruli and blood pressure. Less of one, more the other? Am. J. Hypertens. 1, 335-347.

Fetterman, G. H. & Habib, R. (1969) Congenital bilateral oligonephronic renal hypoplasia with hypertrophy of nephrons (oligoméganéphronie). Am. J. Clin. Path. 52, 199-207.

Yoshida, Y., Fogo, A., & Ichikawa, I. (1989) Glomerular hemodynamic changes vs. hypertrophy in experimental glomerular sclerosis. Kidney Int. 35, 654-660.

Terzi, F. Ticozzi, C., Burtin, M., Motel, V., Beaufils, H., Laouari, D., Assael, B. M., & Kleinknecht, C. (1995) Subtotal but not unilateral nephrectomy induces hyperplasia and protooncogene expression. Am. J. Physiol. 268, F793-F801.

Floege, J., Burns, M. W., Alpers, C. E., Yoshimura, A., Pritzl, P., Gordon, K., Seifert, R. A., Bowen-Pope, D. F., Couser, W. G., & Johnson, R. J. (1992) Glomerular cell proliferation and PDGF expression precede glomerulosclerosis in the remnant kidney model. Kidney Int. 41, 297-309.

Fries, J., Sandstrom, D., Meyer, T. & Rennke, H. (1989) Glomerular hypertrophy and epithelial cell injury modulate progressive glomerulosclerosis in the rat. Lab. Invest. 60, 205-218.

Goss, R. J. (1966) Hypertrophy versus hyperplasia. Science 153, 1615-1620.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method for treating or preventing a pathophysiological state of an organ in an individual wherein said state is characterized by an undesirable level of cyclin-dependent kinase inhibitor activity in said organ, comprising the step of regulating the expression of the p21 gene in said organ of said individual.

2. The method of claim 1, wherein said organ is selected from the group consisting of kidney, heart, liver, and lung.

3. The method of claim 1, wherein said pathophysiological state is selected from the group consisting of renal fibrosis, glomerulosclerosis, reduced filtration rates, hypertension, and organ transplantation rejection.

4. The method of claim 1, wherein the regulation of the expression of the p21 gene results in the reduction or elimination of the expression of the p21 gene.

5. The method of claim 4, wherein the reduction or elimination of the expression of the p21 gene is performed by a technique selected from the group consisting of drug therapy, and genetic manipulation.

6. A method for treating or preventing chronic progressive renal failure in an individual, comprising the step of regulating the expression of the p21 gene in one or both kidneys of said individual wherein said regulation of the p21 gene results in the manipulation of cyclin-dependent kinase inhibitor activity in one or both kidneys.

7. The method of claim 6, wherein the regulation of the expression of the p21 gene results in the reduction or elimination of the expression of the p21 gene.

8. The method of claim 7, wherein the reduction or elimination of the expression of the p21 gene is performed by a technique selected from the group consisting of drug therapy, and genetic manipulation.

9. A method of lowering the rate of long-term rejection of a transplanted organ in an individual comprising the step of transplanting into said individual the organ from a donor wherein the p21 gene in said organ is not expressed.

10. The method of claim 9, wherein said organ is selected from the group consisting of kidney, heart, liver, and lung.
